(19) United States
(12) Patent Application Publication
(10) Pub. No.: US 2012/0114758 A1
(43) Pub. Date: May 10, 2012

(54) BEVERAGE FOR PREGNANCY & LACTATION

(76) Inventor: James Elgin Rea, Eden Prairie, MN (US)

(21) Appl. No.: 13/095,034

(22) Filed: Apr. 27, 2011

Related U.S. Application Data
(60) Provisional application No. 61/355,708, filed on Jun. 17, 2010.

(51) Int. Cl. A61K 33/26; A61P 15/14; A61P 3/02; A61K 9/14; A61K 33/30

(52) U.S. Cl. 424/490; 424/646; 424/643

(57) ABSTRACT

The invention generally relates to nutrient enhanced beverages which include certain levels of the most current Daily Recommended Intakes of macronutrients and Micronutrients, and the rationale for increased allowances during pregnancy. The beverages of the invention generally comprise one or more macronutrients, vitamins and minerals recommended for consumption by pregnant or lactating women in an amount that is effective for enhancing the nutrition of pregnant and lactating women, and that is not harmful to developing fetuses or breast-feeding babies. In particular, the invention provides high doses of more easily absorbable iron to increase bioavailability of both iron and calcium which normally are not ingested in the needed doses, or are usually ingested via hard to swallow and digest prenatal, iron and calcium pills that can cause stomach cramps, diarrhea, constipation and other gastro-intestinal effects, that often lead to women not getting enough necessary nutrients.

### RELATED APPLICATIONS

The present application is a continuation application of U.S. provisional patent application, Ser. No. 61/355,708, filed Jun. 17, 2010, for NUTRIENT ENHANCED BEVERAGE FOR PREGNANCY & LACTATION TO ADDRESS NUTRIENT DEFICIENCIES, by James E Rea, included by reference herein and for which benefit of the priority date is hereby claimed.

The present application is related to U.S. Pat. No. 4,710,387, issued Nov. 7, 1985, for NUTRITIONAL SUPPLEMENT PREPARATION INTENDED FOR PREGNANT AND BREAST-FEEDING WOMEN BASED ON MILK CONSTITUENTS AS WELL AS A PROCESS FOR PREPARING IT, by Uiterwaal; Dirk J. D., Melkunte Holland B. V., included by reference herein.

The present application is related to U.S. Pat. No. 4,751,085, issued Jun. 14, 1988, for HUMAN NUTRITIONAL COMPOSITIONS CONTAINING TAURINE AND VITAMINS AND/OR MINERALS, by Gaull; Gerald E., included by reference herein.

The present application is related to U.S. Pat. No. 5,494,678, issued Feb. 27, 1996, for MULTI-VITAMIN AND MINERAL SUPPLEMENT FOR PREGNANT WOMEN, by Paradissis, George, included by reference herein.

The present application is related to U.S. Pat. No. 5,571,441, issued Nov. 5, 1996, for NUTRIENT SUPPLEMENT COMPOSITIONS PROVIDING PHYSIOLOGICAL FEEDBACK, by Andon, Mark B., included by reference herein.

The present application is related to U.S. Pat. No. 5,985,339, issued Nov. 16, 1999, for REFRIGERATION-SHELF-STABLE READY-TO-DRINK COMPLETE NUTRITIONAL COMPOSITIONS AND PRODUCTS, by Kamarei; A. Reza, included by reference herein.

The present application is related to U.S. Pat. No. 6,039,978, issued Mar. 21, 2000, for DIETARY FOOD ENHANCEMENT AGENT, by Bangs; William E. , Khoo; Chor San Heng, Ko; Sandy, included by reference herein.

The present application is related to U.S. Pat. No. 6,569,445, issued Apr. 27, 2003, for FOOD BARS CONTAINING NUTRITIONAL SUPPLEMENTS AND ANTI-CONSTIPATION AND REGULARITY MAINTAINING-AGENTS, by Manning; Paul B., included by reference herein.

The present application is related to U.S. Pat. No. 6,576,253, issued Jun. 10, 2003, for FOOD BARS CONTAINING NUTRITIONAL SUPPLEMENTS, by Manning; Paul B., included by reference herein.

The present application is related to U.S. Pat. No. 7,390,509, issued Jun. 24, 2008, for COMPOSITIONS AND METHODS FOR NUTRITION SUPPLEMENTATION, by Giordano; John A., Balzer; Charles, included by reference herein.

The present application is related to U.S. Pat. No. 7,560,123, issued Jul. 14, 2009, for COMPOSITIONS AND METHODS FOR NUTRITION SUPPLEMENTATION, by Giordano; John A., Balzer; Charles J., included by reference herein.

The present application is related to U.S. Pat. No. 7,585,527, issued Sep. 8, 2009, for COMPOSITION AND METHOD FOR TREATING IRON DEFICIENCY ANEMIA, by Venkataraman; Bala, Guthrie; Michael, included by reference herein.

### FIELD OF THE INVENTION

The present invention relates generally to nutrient enhanced beverages and more specifically it relates to a nutrient enhanced beverage for pregnancy and lactation to address nutrient deficiencies for the purpose of addressing the inherent issues which pregnant and lactating women experience in achieving proper nutrition principally due to a reliance on hard to swallow, digest and absorb prenatal, iron and calcium pills that can cause stomach cramps, diarrhea, constipation and other gastro-intestinal effects that often lead to women not getting enough necessary nutrients. In particular, the present invention relates to beverages comprising macronutrients, minerals and vitamins recommended for consumption by pregnant and lactating women in amounts that are effective for enhancing the nutrition of the woman or their developing fetuses or babies, and methods for improving digestion and bioavailability of key nutrients Calcium and Iron by the inclusion of both iron chelate or microencapsulated iron and elemental or chelated calcium.

### BACKGROUND OF THE INVENTION

Pregnancy is the one of the most nutritionally demanding times of a woman's life, with puberty not far behind. Her body needs enough nutrients every day to support the growth of her baby and the maintenance of and changes in her own body. All the nourishment her developing baby needs comes from her, either through the foods she eats or the supplements she takes. Subsequent to delivering her baby, nursing continues to draw on the resources of her body. And unfortunately, one of the most common comments heard from moms and pregnant women is that “pregnancy is the time in your life when you can eat anything you want.” Usually referring to the number of additional calories needed on top of her regular diet. Unfortunately, that's just not quite true. What she eats matters a whole lot.

Generally speaking, a nutrient is anything that can be used by a living thing to provide energy and build tissue; nutrients support growth, repair and movement. Nutrients are mostly not produced by the body, with the notable exception of certain fats, Vitamin D which can come from exposure to sunlight, Vitamin K which is made by bacteria in the intestines or cholesterol which is produced internally by the liver. Or at least not in enough quantity to be useful. They have to be taken in externally. They actually break down into a few simple categories:
Nutrients that provide calories for energy, like carbohydrates, protein and fats.
Nutrients that support growth and repair, which include proteins, minerals and vitamins.
Fiber which enhances digestive tract performance regulates blood sugar levels and helps to eliminate circulating levels of LDL (“bad”) cholesterol.
Non-nutrient ingredients that help bind the sources of this stuff all together and help form the taste, aroma and texture of what we eat. Not to mention the ingredients and processes to keep food from spoiling by the time it hits the store shelves.

The most important thing to know about all of these is that they are dose-dependent. In other words, how much of them she consumes determines how well they support growing, maintaining and powering her and her baby. And the fact that in some instances too much can be as harmful as too little.

The Daily Recommended Intakes (DRI) of nutrients (carbohydrates, fiber, protein, fats, vitamins and minerals) for 22 life stages including pregnancy and nursing by age group are defined and refined periodically by the Institute of Medicine of the National Academies (IOM). There are levels for infants, children, males, females, pregnancy and lactation, by age range. For each nutrient, the IOM establishes several levels, but the ones we will focus on are the Daily Recommended Intakes (DRI) and the Tolerable Upper Intake Level (UL). The Institute of Medicine of the National Academies (IOM) is an independent, nonprofit organization that works outside of government to provide unbiased and authoritative advice to decision makers and the public on health and health care. They provide research and answers to Congress, federal agencies like the FDA and others, on topics such as the level of nutrients needed for good health. The most recent DRI levels, established in the early to mid 2000's and on-going, are described in detail in the “Dietary Reference Intakes—Guiding Principles for Nutrition Labeling and Fortification” documents available from the IOM. Prior to the most current Guides, the IOM used different measurement systems, units of measure and levels. We use the current measurement systems, units of measure and levels and it should be assumed that this patent is valid for any future changes to those items. Additionally, the Food and Drug Administration defines Daily Recommended Allowances (otherwise shown on food labels as “Daily Values”) which were established over several decades and are currently out of date and inconsistent with current science. The present invention is based on the most current IOM DRI levels.

Daily Recommended Intakes (DRI) is essentially defined by the IOM as “an estimate of the minimum daily average dietary intake level that meets the nutrient requirements of nearly all (97 to 98 percent) healthy individuals in a particular life stage and gender group” or, if insufficient science exists to be that certain, the best experiential estimate of the level needed. More clearly, it's the IOM's most current understanding of how much an average individual needs to consume at a minimum on average each day of each nutrient. The amount actually consumed can vary substantially from day to day without ill effect in most cases, but on average for an average sized person, these are the levels that are best known to be consumed.

Tolerable Upper Intake Level (UL) is defined as “the highest average daily nutrient intake level that is likely to pose no risk of adverse health effects to almost all individuals in the general population. As intake increases above the UL, the potential risk of adverse effects may increase.”

Not all nutrients must be taken in externally since the body stores enough of them, should get as little as reasonably possible, or are produced within the body. Those nutrients in FIG. 1 that have a check mark must be consumed from external resources. Typically, pregnant and nursing women obtain the checked DRI of macronutrients (carbohydrates, fiber, protein and fats) from foods. They will often obtain the DRI of Minerals and Vitamins through foods, prenatal vitamins, and additional supplements such as iron and calcium pills.

As used herein, the term “pill” can refer to any non-food format of a mineral or vitamin including but not limited to pills, chews, tablets, caplets, lozenges, gels, syrups, sprinkles, powders or any other similar form factor. “Chelated” or “Microencapsulated” may refer to any form of technology used to increase bioavailability and/or reduce gastrointestinal issues caused by consumption of various forms of iron or calcium.

Calcium is a fundamental component of bone and teeth development, and helps prevent blood clots as well as helps muscles and nerves function. Both the pregnant and nursing woman and her baby need sufficient calcium to maintain health.

Women need extra calcium throughout pregnancy and nursing. In fact, if they do not provide enough from external sources their body taps into the calcium in their own bones. While over time this bone calcium can be replaced, it also can lead to osteoporosis later in life. Absorption of calcium decreases the larger the dose, so usually it is best to consume less than 500 mg (½ the UL) at one time. In addition, calcium interferes with iron absorption, so should not be taken in elemental form in large doses with most forms of iron. That is one of the reasons that prenatal vitamins typically include 20-25% of the Calcium DRI. As well, packing the DRI of 1,000 mcg into a single pill would make a pill that is very hard to swallow and would not be fully digested.

Most women don't get the DRI of Calcium even during non-pregnancy, so the need for supplementation is even greater during pregnancy and nursing. Usually women will need to supplement their calcium intake even if they are taking a prenatal vitamin. Usually, if taking calcium supplements it is best to space them out throughout the day to make sure her body can absorb them as much as possible.

Calcium pills are often hard to swallow, taste awful and are hard to digest. For these reasons and others, pregnant and lactating women often don't ingest the needed calcium levels.

Iron helps women's bodies make new blood to carry the oxygen and nutrients to the baby during pregnancy and is needed for the developing blood supply of the baby. By the end of pregnancy there is twice as much blood in the body than when pregnancy began. The need for iron will increase 50% over pre-pregnancy requirements. Pregnant and lactating women will need to take extra iron because their normal diet usually will not provide the required amount. About 20% of women, 50% of pregnant women, and 3% of men do not have enough iron in their body.

The United States Center for Disease Control (CDC) recommends universal iron supplementation to meet the iron requirements of pregnancy. The CDC recommendations for iron supplementation during pregnancy are similar to the guidelines issued by the American Academy of Pediatrics and the American College of Obstetricians and Gynecologists. Primary prevention of iron deficiency during pregnancy includes both adequate dietary iron intake and iron supplementation.

Supplemental iron pills often cause diarrhea, constipation, upset stomach, prevent the body from taking in other nutrients, and are not as readily absorbed as iron from food. The amount of iron absorbed decreases with increasing doses. For this reason, if use of iron pills is recommended, often people take their prescribed daily iron supplements in two or three equally spaced doses. Taking iron with Vitamin C can help with the absorption of iron.

Iron pills are often hard to swallow, taste awful and are hard to digest. For these reasons and others, pregnant and lactating women often don't ingest the needed iron levels.

Many prenatal vitamin and iron pills contain “elemental” iron (essentially iron filings) or iron salts. Essentially like swallowing the metal raw. These are very hard to digest, but they are cheap. The safety of iron salts during pregnancy and nursing is unclear. Iron salts go by various names such as ferrous sulfate, ferrous fumarate, and ferrous gluconate. The FDA generally says of these salts “There is no evidence in the available information on [this form of iron] that demonstrates a hazard to the public when it is used at levels that are now current and in the manner now practiced. However, it is not possible to determine, without additional data, whether a significant increase in consumption would constitute a dietary hazard.”

Certain products are made with more expensive “micro-encapsulated” or “chelated” iron which are mostly absorbed lower in the intestine. These forms of iron have little iron flavor, cause far less gastrointestinal issues, and are more easily absorbed by the body than elemental iron or iron salts. At least one form of microencapsulated iron and one form of iron chelate is considered by the FDA as Generally Recognized as Safe.

Dietary Fibers are low in energy because they aren't digestible. Although that means they are not much of a source of energy, fibers can help slow down digestion overall, moderating the overall level of blood sugar. They have other uses, in particular for pregnancy. Specific to the present invention they may help decrease common symptoms such as constipation and hemorrhoids. The DRI for fiber during pregnancy and nursing is 28-29 g per day. The average American eats approximately 10 g per day. Pregnant women need to increase fiber intake. Increasing fiber will also require an increase in liquid intake to avoid constipation, which is provided by the beverage.

The DRI numbers for each nutrient are defined. But just because a pill contains perhaps 27 mg of iron, does not mean that the person ingesting it will actually get 27 mg into their system. For example, suppose a person swallowed an iron toy marble. The entire marble would not be digested, partly because the digestive tract would push it through far faster than that much iron could be broken down and absorbed. The same applies to nutrients like Calcium and others. Calcium is slow to digest, swallow and interacts to make iron less digestible. So prenatal vitamins contain far less Calcium than is actually needed.

The amount of a nutrient that is absorbed and made available to the body is referred to as its Bioavailability. Drugs that are injected intravenously are automatically 100% bioavailable, since they are fully delivered into the blood stream. Nutrients that are taken orally generally are less than 100% bioavailable. Many factors determine how bioavailable an oral nutrient is including:
Physical properties and overall digestibility of the nutrient itself
Interaction with other drugs or nutrients
How fast the stomach empties
How full the stomach was
The health of the Gastrointestinal tract
How the nutrient interacts with enzymes in the gastrointestinal system
Age, and others

So prenatal vitamins aren't enough. It is likely the woman will need either supplements, and for iron or calcium in particular.

Iron and calcium pills often taste awful and can be hard to swallow. Elemental calcium and iron also compete with each other for digestion and absorption. If ingesting high levels of them together, typically their combined bioavailability is very low. In addition, because they are minerals that the stomach must break down and restructure in a way that can be absorbed in the blood stream. They can cause stomach cramps, constipation and other gastro-intestinal issues. Women sometimes simply stop taking these supplements for these reasons.

Most iron supplements today use cheap iron salts and calcium supplements usually use cheap calcium carbonate, both of which are digested and absorbed in the stomach and upper intestine. It is this digestion and absorption that causes the gastro-intestinal effects and conflict between the absorption of the two minerals together.

Per FIG. 2, products that use more expensive iron chelates or microencapsulated iron don't require iron digestion, and are absorbed lower in the intestine, often after the bile and pancreatic ducts. Since these iron forms are already in a readily absorbable form, and absorption occurs lower in the intestine, gastro-intestinal issues are significantly reduced, absorption is higher, and the minerals compete less with each other. Per the comparison of calcium and iron absorption with elemental minerals versus chelated and microencapsulated iron, calcium is digested in the stomach and absorbed mostly higher in the intestine. Iron chelate and microencapsulated irons are not digested and are absorbed further along the intestine, thus calcium and iron compete less with each other for absorption, raising their bioavailabilities.

Many partial solutions exist today. For instance, prenatal vitamins are recommended for all pregnant women. Many women stop taking prenatals because of the related stomach cramps and constipation. Prenatals, due to the slow absorption and what would be a pill too large to swallow, only include about 20-25% of daily required calcium intake and must be supplemented with hard to swallow and digest pills. Other pills are available for iron, but typically provide elemental iron that causes stomach cramps and constipation, often leading the woman to stop taking those pills. Only one product in the United States that we are aware of attempts to provide supplemental nutrients via foods to pregnant and nursing women, through snack bars and chews. These products use elemental iron if any, and are not available in liquid form. They are not formulated to address bioavailability or gastrointestinal issues.

Conventional prenatal nutrient supplements are typically limited in scope. They contain a subset of nutrients needed for pregnancy and nursing. In addition, none deal with the bioavailability of calcium and iron (which conlict with each other for digestion and absorption), do not address the gastrointestinal issues caused by typical iron supplements, do not address the fiber needed to assist with steady energy and constipation, and do not address bioavailability of all vitamins and nutrients by spreading their absorption throughout the day in beverage format. No complete soluton is availabe to address all of the nutritional needs in conventient format that is delicious and thus encourages intake of these nutrients.

An object is to provide a nutrient enhanced beverage for pregnancy and lactation to address nutrient deficiencies for the purpose of addressing the inherent issues which pregnant and lactating women experience in achieving proper nutrition during pregnancy and lactation principally due to a reliance on hard to swallow and digest prenatal, iron and calcium pills that can cause stomach cramps, diarrhea, constipation and other gastro-intestinal effects that often lead to women not getting enough necessary nutrients, and methods for supplementing these dietary requirements of pregnant or lactating women by the inclusion of both iron chelate or microencapsulated iron and elemental calcium or chelated calcium.

Another object is to provide a nutrient enhanced beverage for pregnancy and lactation to address nutrient deficiencies that incents its use by the nature of its delicious flavor. Pregnant women need to ingest 0.8 gallons of liquids per day. Lactating women need to ingest 1 gallon of liquids. By focusing on delicious flavors, women will find the invention an attractive way to achieve their needed daily fluid intake, as well as benefiting from its unique nutrient and physiological benefits. By including key minerals (iron and calcium) in a beverage, their absorption into the body is spread out throughout the day, increasing bioavailability.

Another object is to provide a nutrient enhanced beverage for pregnancy and lactation to address nutrient deficiencies that provides an appropriate balance of Vitamin C, D, Calcium and Iron to assist with their bioavailability.

Another object is to provide a nutrient enhanced beverage for pregnancy and lactation to address nutrient deficiencies that provides an appropriate level of dietary fiber to assist with gastro-intestinal issues.

Another object is to provide a nutrient enhanced beverage for pregnancy and lactation to address nutrient deficiencies that provides more easily absorbable iron chelate or microencapsulated iron and elemental calcium or chelated calcium to improve their combined bioavailability, avoid gastrointestinal problems, and avoid digestion and absorption conflicts since elemental calcium and iron negatively affect each others bioavailability.

Another object is to provide a nutrient enhanced beverage for pregnancy and lactation to address nutrient deficiencies that provides appropriate levels of protein and carbohydrates for steady energy, healthy weight gain and overall growth and maintenance.

### SUMMARY OF THE INVENTION

The invention generally relates to nutrient enhanced beverages which include certain levels of the most current Daily Recommended Intakes of macronutrients and micronutrients, and the rationale for increased allowances during pregnancy and lactation, as established by the Institute of Medicine of the National Academies (IOM). The beverages of the invention generally comprise one or more macronutrients, vitamins and minerals recommended for consumption by pregnant or lactating women in an amount that is effective for enhancing the nutrition of pregnant and lactating women, and that is not harmful to developing fetuses or breast-feeding babies, absorbable minerals which increase their bioavailability, and that are not harmful to developing fetuses or breast-feeding babies.

In particular, the invention provides high doses of more easily absorbable iron to increase bioavailability of both iron and calcium which normally are not ingested in the needed doses, or are usually ingested via hard to swallow and digest prenatal, iron and calcium pills that can cause stomach cramps, diarrhea, constipation and other gastro-intestinal effects, that often lead to women not getting enough necessary nutrients. Additionally appropriate levels of protein, fiber, and essential vitamins and vitamins necessary for brain, body, bone and blood development are included per current IOM science.

Before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a detail view of the most current dri and ul levels for all nutrients, for pregnancy and nursing for ages 19-50 with those Minerals and Vitamins typically gotten from supplementation during pregnancy marked with a check; and

FIG. 2 is an explanatory view comparing the digestion and absorption of various forms of calcium and iron.

For purposes of clarity and brevity, like elements and components will bear the same designations and numbering throughout the Figures.

### DESCRIPTION OF THE PREFERRED EMBODIMENT

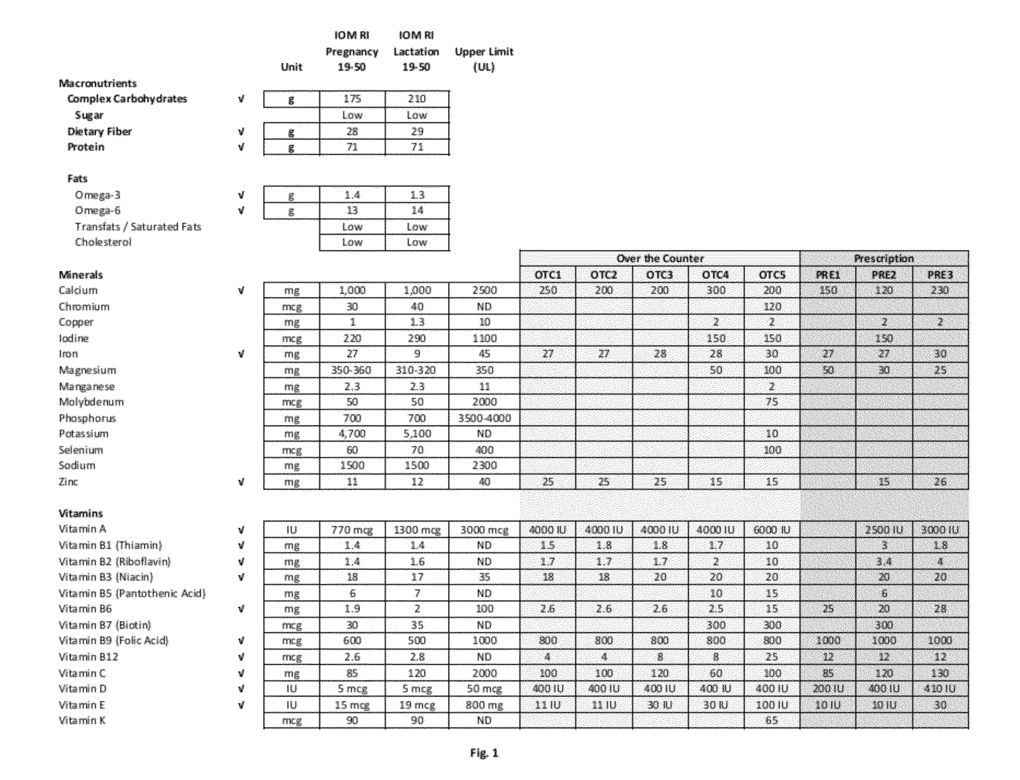

FIG. 1 details the nutrients which pregnant and lactating women need be concerned with, based on the most current scientific analyses and published results from the Institute of Medicine of the National Academies. For the most part, prenatal vitamins are not intended to provide macronutrients. Food, including beverages as food, are.

In FIG. 1 we have included the Pregnancy and Lactation DRI and UL levels from the IOM for women aged 19-50 as well as comparison to representative ingredient levels of common Over The Counter (“OTC”) and prescription (“PRE”) prenatal vitamins which have been used to establish the present inventions nutrient ranges.

Health care providers often recommend that women take a daily prenatal vitamin, and perhaps other supplements. Sometimes they will recommend a prescription prenatal vitamin.

A wide variety of nutrients that are safe for consumption by pregnant women or lactating women may be used in the beverages of the invention in varying quantities. The beverages of the invention may be formulated using any pharmaceutically-acceptable forms of the nutrients described below, including their salts, which are known by those of skill in the art. The nutrients that are employed in the beverages of the invention are those that are recommended for consumption by pregnant women and lactating. These nutrients are employed in an amount that is effective for enhancing the nutrition of pregnant women and lactating women, or of their developing fetuses or babies.

Each beverage may contain one or more of the nutrients in FIG. 1 in any quantity that is safe for consumption by pregnant women and lactating women (i.e., a quantity that would not cause harm to the woman consuming the beverage, or to her developing fetus or breast-feeding baby). It should be noted that some nutrients may be set to a range including zero units. This is due to the fact that the stability of microencapsulated items may vary depending on the method of microencapsulation used by the supplier. It is not possible to provide in this patent all possible combinations of matter that might conflict with all known microencapsulated items. Persons knowledgeable in the art will know how to test for compatibility.

Some definitions:

Unit—the unit of measure being used for the nutrient.
g=gram.
mg=milligram (1,000th of a gram).
mcg=microgram (1,000th of a mg).

IU is an old measure called international units. IU's don't convert directly to gram-based units because they vary by the source of the nutrient.

The beverages of the invention generally comprise one or more macronutrients, vitamins and minerals recommended for consumption by pregnant or lactating women in an amount that is effective for enhancing the nutrition of pregnant and lactating women, and that is not harmful to developing fetuses or breast-feeding babies, highly absorbable minerals which increase their bioavailability, and that are not harmful to developing fetuses or breast-feeding babies. Prior art for pregnancy and lactation has relied on elemental iron or iron salts as well as elemental calcium. The present invention relies on chelated iron or microencapsulated iron and elemental or chelated calcium.

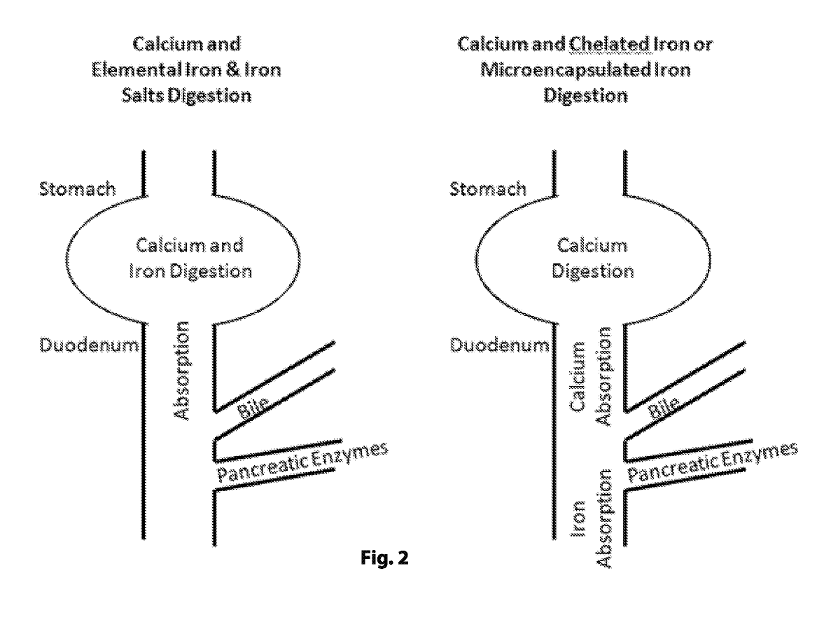

FIG. 2 diagrams the digestion and absorption of various forms of calcium and iron. When using elemental iron or iron salts with elemental calcium, the iron and calcium must both be broken down in the stomach during the “Gastric phase” of digestion. This phase takes 3 to 4 hours. It is stimulated by distention of the stomach, presence of food in stomach and decrease in pH. Distention activates long and myentric reflexes. This activates the release of acetylcholine which stimulates the release of more gastric juices. As protein enters the stomach, it binds to hydrogen ions, which lowers the pH of the stomach to around pH 1-3. Inhibition of gastrin and HCl secretion is lifted. This triggers G cells to release gastrin, which in turn stimulates parietal cells to secrete HCl. HCl release is also triggered by acetylcholine and histamine. When both iron and calcium are in their elemental or salt forms, they compete with each other for digestion in the Gastric phase. Thus, not all of the minerals are broken down for absorption.

During the next “Intestinal phase” partially digested food fills the duodenum. This triggers intestinal gastrin to be released. Enterogastric reflex inhibits vagal nuclei, activating sympathetic fibers causing the pyloric sphincter to tighten to prevent more food from entering, and inhibits local reflexes. Most calcium and iron absorption occurs in the duodenum. Thus, again the calcium and iron conflict with each other for absorption.

When chelated Iron or microencapsulated iron are used with Calcium, the calcium alone is digested in the stomach. Since the iron has already been broken down it is ready for absorption into the blood stream, little conflict exists between iron and calcium digestion in the stomach. Chelated iron and microencapsulated iron pass through the duodenum and into the intestine below the bile and pancreatic enzyme ducts, where it is more readily absorbed and conflicts less with calcium absorption.

Because of this, both iron and calcium are more highly absorbed, with far less gastro-intestinal issues than in prior art.

In particular, the invention provides high doses of chelated iron or microencapsulated iron and elemental calcium or chelated calcium to increase bioavailability of these critical minerals which normally are not ingested in the needed doses, or are usually ingested via hard to swallow and digest prenatal, iron and calcium pills that can cause stomach cramps, diarrhea, constipation and other gastro-intestinal effects, that often lead to women not getting enough necessary nutrients. The invention specifies that both iron chelate or microencapsulated iron and elemental calcium or chelated calcium be included as key nutrients, something not specified in prior art. Additionally appropriate levels of protein, fiber, and essential vitamins and vitamins necessary for brain, body, bone and blood development are included per current IOM science.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A beverage for pregnancy & lactation to address nutrient deficiencies, comprising:
about 50 to 500 mg (elemental equivalent) calcium—chelated or elemental;
about 3 to 27 mg (elemental equivalent) iron—chelated or microencapsulated.

2. The beverage for pregnancy & lactation as recited in claim 1, further comprising about 1 to 29 g dietary fiber.

3. The beverage for pregnancy & lactation as recited in claim 1, further comprising:
between zero and their UL, other vitamins and minerals, for appropriate nutrition without exceeding UL's, of vitamins and minerals that either need daily intake, or which support brain, bone, body and blood development. These include zinc, and vitamins A, B1, B2, B3, B5, B6, B9, B12, C, D and K.

4. The beverage for pregnancy & lactation as recited in claim 1, wherein the calcium and/or iron terms “Chelated” or “Microencapsulated” may refer to any form of technology used to increase bioavailability and/or reduce gastrointestinal issues caused by consumption of various forms of iron or calcium.

5. The beverage for pregnancy & lactation as recited in claim 1, further comprising of flavoring and masking agents to provide a delicious beverage.
